(19) United States
(12) Patent Application Publication
(10) Pub. No.: US 2026/0248651 A1
(43) Pub. Date: Aug. 27, 2026

(54) SNOW GOGGLES DEVICE AND MANUFACTURING METHOD THEREOF HAVING A PASSIVELY DEFROSTING THIH FILM

(71) Applicant: FORESIGHT OPTICAL LTD., Tainan (TW)

(72) Inventors: TIEN-SHU WU, Tainan (TW); YEN-TING WU, Tainan (TW)

(21) Appl. No.: 19/187,824

(22) Filed: Apr. 23, 2025

(30) Foreign Application Priority Data
Feb. 27, 2025 (TW) 114107487

(51) Int. Cl. A61F 9/02

(52) U.S. Cl. CPC A61F 9/022; A61F 9/029

(57) ABSTRACT

A manufacturing method of a snow goggles device includes: providing an optical substrate layer on a snow goggles frame; providing the optical substrate layer with a first surface and a second surface, and forming at least one transparent conductive film on the first surface, the second surface or therebetween; electrically connecting the at least one transparent conductive film with a power supply unit and a switch unit, with providing the power supply unit and the switch unit on the snow goggles frame; and operating the power supply unit to supply a current to the at least one transparent conductive film via the switch unit to thereby heat the optical substrate layer.

### BACKGROUND OF THE INVENTION

### 1. Field of the Invention

The present invention relates to a snow goggles device and manufacturing method thereof having a passively defrosting thin film. Particularly, the present invention relates to the snow goggles device and manufacturing method thereof having a passively defrosting thin film on a surface of glasses. More particularly, the present invention relates to the snow goggles device and manufacturing method thereof having a passively defrosting thin film directly heating the surface of glasses.

### 2. Description of the Related Art

Chinese Utility-Model Patent Publication No. CN-211985900, entitled “Goggles,” discloses a goggles device, including a goggles frame, a desiccant compartment, a transparent heating glasses plate and a power supply which has a battery socket and a battery unit mounted therein.

As described above, the goggles frame has an inner wall on which to mount the desiccant compartment. Provided between the goggles frame and the desiccant compartment is a socket-plug connection arc support structure which is a mutually coupled and detachable arc support structure.

As described above, the goggles frame is assembled with the transparent heating glasses plate which is provided with a sandwiched film layer and a heating wire formed therewith. The heating wire heating wire electrically connects with the power supply which has a control switch.

As described above, the goggles frame further includes an exterior power supply connection hole which is provided with a USB interface. The goggles frame further includes a thermal sensor which is electrically connected with a thermal adjustment knob.

However, the goggles frame has the transparent heating glasses plate which is directly heated by the heating wire formed in the sandwiched film layer. Disadvantageously, the heating wire causes interference of vision through the transparent heating glasses plate such that there is a need of improving the design of transparent heating glasses plates of goggles device.

Another Canadian Patent Application Publication No. CA-2,327,006, entitled “Anti-frost goggles and method for manufacturing the same,” discloses an anti-frost goggles device and a manufacturing method thereof. The anti-frost goggles device has a goggles body performed as a frame.

As described above, the goggles body has an inner surface on which to provide a heating element for heating thereon. The heating element further includes a pair of electrodes also provided at the inner surface of the goggle body where has upper and lower portions to provide each of electrodes.

As described above, the pair of electrodes electrically connects with a terminal via a pair of power supply lines and a pair of external wires. The terminal is connected between power supply means and the power supply lines via the external electric wires. A power switch is coupled between the terminal and the power supply means.

However, the heating element is only provided to heat the inner surface of the goggles body. Disadvantageously, the heating element cannot directly heat a glasses lens of the anti-frost goggles device such that there is a need of improving the design of heating for the anti-frost goggles device.

Another Taiwanese Patent Publication No. TW-336691, entitled “Edge coated glass polarized lens,” discloses a glasses polarization lens device formed with an adhesive-coated side edge. The glasses polarization lens device includes a first glasses layer, a second glasses layer, a polarization layer and an adhesive-coated side edge layer.

As described above, the first glasses layer has a first optical surface while the second glasses layer has a second optical surface opposite to the first optical surface. The polarization layer is provided between the first glasses layer and the second glasses layer. The adhesive-coated side edge layer is formed on a peripheral side edge connected between the first glasses layer and the second glasses layer.

As described above, the adhesive-coated side edge layer is formed by polymerization chain reaction of free radicals of UV adhesive material exposing or curing in UV light. The UV adhesive material includes aliphatic polyesterpolyol based urethane methacrylate, a reactive diluent, an adhesion promoter and a photo-initiator. The aliphatic polyesterpolyol based urethane methacrylate is selectively made of aliphatic polyester polyol, methyl methacrylate (MMA) and di-isocyanate and has an average mass ranging between 800 and 4,000.

As described above, the reactive diluent is selected from methyl methacrylate monomer and the adhesion promoter is made of methacrylate-functional silane, amino methacrylate-functional silane, vinyl-functional silane and combinations thereof. The photo-initiator is made of alpha-hydroxy ketone, alpha-amino ketone, benzil dimethyl ketal, benzophenone, phosphine oxide and combinations thereof.

Another U.S. Pat. No. 4,076,863, entitled “Process for cutting and edging chemically pre-strengthened finished uncut lens blanks without loss of impact resistance,” also discloses an edging chemically pre-strengthened finished uncut lens blank which has a side peripheral surface and a side coated layer formed thereon.

As described above, the side coated layer is selectively made of epoxy resin, alkyd resin, polyurethane resin, unsaturated polyester resin, acrylic resin, cyanoacrylate and combinations thereof.

Another U.S. Pat. No. 5,220,358, entitled “Edge coating for laminated lenses,” also discloses an edge-coated, laminated lens device. The edge-coated, laminated lens device has a side peripheral surface and a side coated layer formed thereon.

As described above, the side coated layer is selected from a dimethyl silicone-coated layer. The side coated layer comprises a dimethyl silicone coblocked with vinyl with crosslinking therein being provided through a platinum-catalyzed addition reaction.

Another U.S. Pat. No. 6,860,600, entitled “Edge coated ophthalmic lenses,” also discloses an edge coated ophthalmic lens device. The edge coated ophthalmic lens device has a side peripheral surface and a side coated layer formed thereon.

As described above, the side coated layer of peripheral edge is coated with an edge coating which strongly bonds to the peripheral edge but that only weakly bonds to an optical surface, and the edge coating is durable. A composition of the side coated layer is selected from UV curable acrylates.

However, there is a need of improving the conventional snow goggles device or structure for providing a passively defrosting snow goggles device and manufacturing method thereof. The above-mentioned patents and patent application publications are incorporated herein by reference for purposes including, but not limited to, indicating the background of the present invention and illustrating the situation of the art.

### SUMMARY OF THE INVENTION

The primary objective of this invention is to provide a snow goggles device and manufacturing method thereof having a passively defrosting thin film. An optical substrate layer provided on a snow goggles frame has a first surface and a second surface, with forming at least one transparent conductive film on the first surface, the second surface or therebetween, with electrically connecting the at least one transparent conductive film with a power supply unit and a switch unit, with providing the power supply unit and the switch unit on the snow goggles frame, with operating the power supply unit to supply a current to the transparent conductive film via the switch unit to thereby heat the optical substrate layer. Advantageously, the snow goggles device of the present invention is successful in preventing or avoiding the optical substrate layer turning into a frozen state which will reduce visibility.

The snow goggles device in accordance with an aspect of the present invention includes:
an optical substrate layer provided with a first surface and a second surface;
a snow goggles frame provided with a predetermined surface, with providing the optical substrate layer on the predetermined surface of the snow goggles frame;
at least one transparent conductive film provided on the first surface the second surface or therebetween;
at least one power supply unit electrically connecting with the at least one transparent conductive film, with providing the power supply unit on the snow goggles frame; and
a switch unit electrically connecting between the at least one transparent conductive film and the power supply unit, with providing the switch unit on the snow goggles frame;
wherein the power supply unit is operated to supply an electric current to the at least one transparent conductive film via the switch unit to thereby heat the optical substrate layer for preventing the optical substrate layer turning into a frozen state.

In a separate aspect of the present invention, the transparent conductive film further includes at least one auxiliary heater unit which is selected from a frame-heating member, a heating wire member, a transparent heating film member or combinations thereof.

In a further separate aspect of the present invention, the auxiliary heater unit is integrally formed on a predetermined portion of the snow goggles frame to form a protruded or recessed portion thereon.

In yet a further separate aspect of the present invention, the snow goggles frame is further provided with a compartment to receive the at least one power supply unit and the switch unit.

In yet a further separate aspect of the present invention, the optical substrate layer is provided with a side peripheral surface between the first surface and the second surface to engage with an auxiliary heater unit.

In yet a further separate aspect of the present invention, the transparent conductive film is made of a transparent conductive material or a mixture of transparent conductive material and thermochromic dye material.

In yet a further separate aspect of the present invention, the transparent conductive film further includes at least one auxiliary heater unit provided with at least one illumination member.

In yet a further separate aspect of the present invention, the power supply unit electrically connects with the at least one illumination member.

In yet a further separate aspect of the present invention, the at least one illumination member is selected from an emergency LED element, a solar-cell illumination element, a florescent reflection element or combinations thereof which is integrally formed on the snow goggles frame.

In yet a further separate aspect of the present invention, the switch unit electrically connects with the at least one illumination member.

The manufacturing method of the snow goggles device in accordance with another aspect of the present invention includes:
providing an optical substrate layer on a snow goggles frame;
providing the optical substrate layer with a first surface and a second surface, and providing at least one transparent conductive film on the first surface, the second surface or therebetween;
electrically connecting the at least one transparent conductive film with a power supply unit and a switch unit, with providing the power supply unit and the switch unit on the snow goggles frame; and
operating the power supply unit to supply a current to the at least one transparent conductive film via the switch unit to thereby heat the optical substrate layer for preventing the optical substrate layer turning into a frozen state.

In a separate aspect of the present invention, the transparent conductive film further includes at least one auxiliary heater unit which is selected from a frame-heating member, a heating wire member, a transparent heating film member or combinations thereof.

In a further separate aspect of the present invention, the auxiliary heater unit is integrally formed on a predetermined portion of the snow goggles frame to form a protruded or recessed portion thereon.

In yet a further separate aspect of the present invention, the snow goggles frame is further provided with a compartment to receive the at least one power supply unit and the switch unit.

In yet a further separate aspect of the present invention, the optical substrate layer is provided with a side peripheral surface between the first surface and the second surface to engage with an auxiliary heater unit.

In yet a further separate aspect of the present invention, the transparent conductive film is made of a transparent conductive material or a mixture of transparent conductive material and thermochromic dye material. In yet a further separate aspect of the present invention, the transparent conductive film further includes at least one auxiliary heater unit provided with at least one illumination member.

In yet a further separate aspect of the present invention, the power supply unit electrically connects with at least one illumination member.

In yet a further separate aspect of the present invention, the at least one illumination member is selected from an emergency LED element, a solar-cell illumination element, a florescent reflection element or combinations thereof which is integrally formed on the snow goggles frame.

In yet a further separate aspect of the present invention, the switch unit electrically connects with the at least one illumination member.

Further scope of the applicability of the present invention will become apparent from the detailed description given hereinafter. However, it should be understood that the detailed description and specific examples, while indicating preferred embodiments of the invention, are given by way of illustration only, since various will become apparent to those skilled in the art from this detailed description.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a block diagram of a snow goggles device having a passively defrosting thin film in accordance with a preferred embodiment of the present invention.

FIG. 2 is a schematic front view of a snow goggles device having a passively defrosting thin film in accordance with a first preferred embodiment of the present invention.

FIG. 2A is a schematic front view of a snow goggles device having a passively defrosting thin film in accordance with another preferred embodiment of the present invention.

FIG. 3 is a schematic side view of a snow goggles device having the passively defrosting thin film in accordance with the first preferred embodiment of the present invention.

FIG. 4 is a flow chart of a manufacturing method of snow goggles device in accordance with a preferred embodiment of the present invention.

FIG. 5 is a block diagram of a snow goggles device having a passively defrosting thin film in accordance with a second preferred embodiment of the present invention.

FIG. 6 is a schematic front view of a snow goggles device having a passively defrosting thin film in accordance with a third preferred embodiment of the present invention.

### DETAILED DESCRIPTION OF THE INVENTION

It is noted that a snow goggles device and manufacturing method thereof having a passively defrosting thin film in accordance with the preferred embodiment of the present invention can be applicable to various goggles devices, including various industrial goggle devices, various ski or snow goggle devices, various gliding goggle devices, various cycling goggle devices, various motorcycle-riding goggle devices, or other outdoor or indoor goggle devices, which are not limitative of the present invention.

FIG. 1 shows a block diagram of a snow goggles device having a passively defrosting thin film in accordance with a preferred embodiment of the present invention. Referring now to FIG. 1, the snow goggles device in accordance with the preferred embodiment of the present invention includes a snow goggles frame 1, an optical substrate layer 2, at least one transparent conductive film (passively defrosting ultra-thin film) 3, at least one auxiliary heater unit 4, a switch unit 101 and a power supply unit 102.

With continued reference to FIG. 1, by way of example, the snow goggles frame 1 is selectively made of a predetermined material to form a predetermined shape, e.g. shapes of industrial goggles, ski goggles or other usage goggles, with selecting the predetermined material from a plastic material, an alloy metal material, a composite material (e.g., carbon fiber) or combinations thereof.

With continued reference to FIG. 1, by way of example, the optical substrate layer 2 is selected from a flat lens or a curved lens, with the curved lens selected from a cylindrical lens, a spherical lens, an ellipsoidal lens or other curved lens. The optical substrate layer 2 can also formed from a single-layer lens, a single-layer composite lens, a double-layer lens (i.e., inner-and-outer combined structure), a double-layer hollow lens (i.e., inner-and-outer spaced structure) or other types of lens.

With continued reference to FIG. 1, by way of example, the transparent conductive film 3 is selectively made of a transparent conductive material or a mixture of transparent conductive material and thermochromic dye material, with the conductive material having a predetermined degree of transparency, conductive and resistance. In another preferred embodiment, the conductive material is selected from cadmium oxide (CdO), zin oxide (ZnO), tin oxide (SnO₂), indium oxide (In₂O₃), indium tin oxide (ITO) or combinations thereof which may be provided in multiple layers of the transparent conductive film 3.

With continued reference to FIG. 1, by way of example, the transparent conductive film 3 has a doped material which is selected from a group III material (e.g., B, Al, Ga, In, Sc), a group IV material (e.g., Si, Ge, Sn, Pb, Ti, Zr, Hf), a fluorine material, a carbon nanotube (CNT) material, a metal nano-particle material, a grapheme material or combinations thereof.

With continued reference to FIG. 1, by way of example, the auxiliary heater unit 4 of another preferred embodiment is selected from a frame-heating member, a heating wire member, a transparent heating film member, with the auxiliary heater unit 4 being also selected from a PTC (positive temperature coefficient) thermistor or other equivalent component.

With continued reference to FIG. 1, by way of example, the switch unit 101 is selected from a switch button unit, a press key switch unit, a touch-panel switch unit, a tag switch unit or other equivalent switch units.

With continued reference to FIG. 1, by way of example, the power supply unit 102 is selected from a lithium-battery unit, a button cell battery unit or other equivalent power supply units. In another embodiment, the power supply unit 102 is further selected from a primary cell unit, a secondary cell unit or other equivalent auxiliary power supply units.

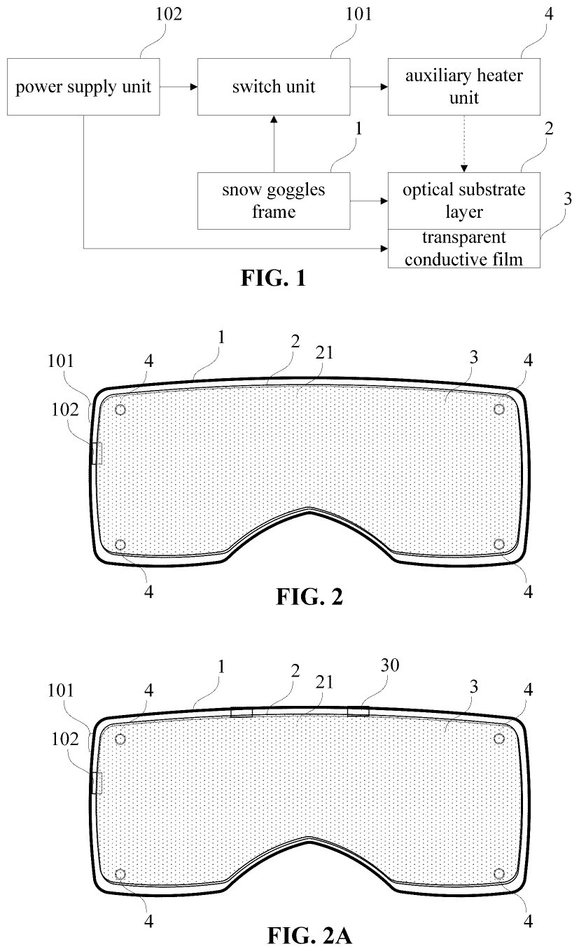

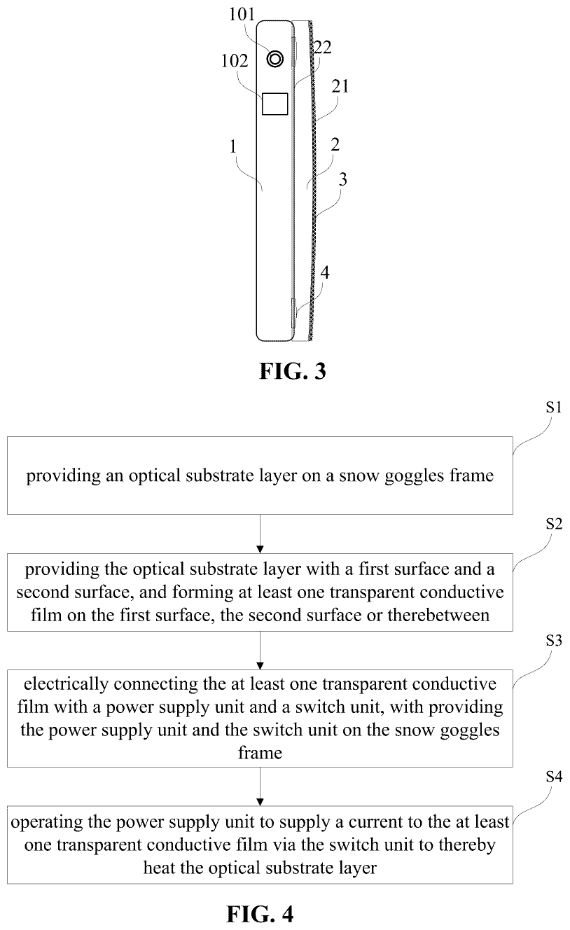

FIG. 2 shows a schematic front view of a snow goggles device having a passively defrosting thin film in accordance with a first preferred embodiment of the present invention. FIG. 2A shows a schematic front view of a snow goggles device having a passively defrosting thin film in accordance with another preferred embodiment of the present invention. FIG. 3 shows a schematic side view of a snow goggles device having the passively defrosting thin film in accordance with the first preferred embodiment of the present invention, corresponding to the snow goggles device shown in FIGS. 2 and 2A.

Referring now to FIGS. 1, 2, 2A and 3, by way of example, the snow goggles frame 1 has a predetermined outline, e.g. outlines of industrial goggles, ski goggles or other usage goggles, a predetermined pattern, a predetermined color or a predetermined strengthen structure. With continued reference to FIGS. 1, 2, 2A and 3, by way of example, the optical substrate layer 2 is formed from a single layer or multiple layers, with the optical substrate layer 2 having a first thickness (i.e. uniform or varying thickness). The optical substrate layer 2 is selected from a glass optical layer, a plastic optical layer, a biodegradable plastic optical layer, a PC (polycarbonate) optical layer, a PMMA (poly(methyl methacrylate)) optical layer, a nylon optical layer, or other similar material layers thereof.

Still referring to FIGS. 1, 2, 2A and 3, by way of example, the optical substrate layer 2 is selected from a polarization thin-film layer, an anti-reflection layer, a photochromic layer, an anti-blue layer, an anti-blue UV layer, an anti-infrared layer, a chroma enhancement layer, other functional thin-film layer (e.g., anti-fog layer or scratch-resistant layer) or combinations thereof.

With continued reference to FIGS. 1, 2, 2A and 3, by way of example, the optical substrate layer 2 has a first surface 21 and a second surface 22, with the first surface 21 performing as a front-side surface (i.e., front outer surface), with the second surface 22 performing as a rear-side surface (i.e., rear inner surface).

With continued reference to FIGS. 1, 2, 2A and 3, by way of example, the snow goggles frame 1 and the optical substrate layer 2 can be selectively made of same materials (e.g., similar materials) or dissimilar materials (e.g., heterogeneous materials) to provide various characteristics of similar or dissimilar materials.

With continued reference to FIGS. 1, 2, 2A and 3, by way of example, the transparent conductive film 3 can be integrally formed on a predetermined portion of the first surface 21 and the second surface 22 of the optical substrate layer 2 or therebetween. Provided between the first surface 21 and the second surface 22 of the optical substrate layer 2 is a side peripheral surface to engage with the transparent conductive film 3. In another preferred embodiment, the transparent conductive film 3 engages with the side peripheral surface between the first surface 21 and the second surface 22 which is preferably provided with a groove to receive the transparent conductive film 3.

With continued reference to FIGS. 1, 2, 2A and 3, by way of example, the auxiliary heater unit 4 (or illumination member) is integrally formed on a predetermined portion of the snow goggles frame 1, including outer surfaces, inner surfaces or others, to form a protruded or recessed portion thereon.

With continued reference to FIGS. 1, 2, 2A and 3, by way of example, the transparent conductive film 3 of another preferred embodiment includes a pair of illumination members 30 (as best shown at left and right in FIG. 2A) electrically connecting with the power supply unit 102, with the illumination members 30 selecting from a LED element, an emergency LED element, a solar-cell illumination element, a florescent reflection element or combinations thereof which electrically connects with the switch unit 101.

With continued reference to FIGS. 1, 2, 2A and 3, by way of example, the transparent conductive film 3 has a perfect degree of transparency, with the transparent conductive film 3 further having a degree of scratch resistance, anti-chemical corrosion, anti-static or flexibility.

FIG. 4 shows a flow chart of a manufacturing method of snow goggles device in accordance with a preferred embodiment of the present invention corresponding to the snow goggles device shown in FIGS. 1-3. Turning now to FIGS. 1 through 4, by way of example, the manufacturing method of snow goggles device has a series of procedure (steps S1, S2, S3 and S4) which may be modified according to needs. The manufacturing method of snow goggles device of the present invention includes the step S1: providing the optical substrate layer 2 on the snow goggles frame 1 by means of assembling, molding, injection molding or other suitable manners, with preprocessing at least one predetermined surface (e.g. front side) of the snow goggles frame 1 to provide a pre-processed area thereon.

Still referring to FIGS. 1 through 4, by way of example, the manufacturing method of snow goggles device of the present invention further includes the step S2: providing or forming the optical substrate layer 2 with the first surface 21 and the second surface 22 by means of polishing or other suitable manners, and providing at least one transparent conductive film 3 on at least one pre-processed area of the first surface 21, the second surface 22 or therebetween by means of sputtering, laser printing, spray coating, shower coating, adhering or other suitable manners.

Still referring to FIGS. 1 through 4, by way of example, the manufacturing method of snow goggles device of the present invention further includes the step S3: electrically connecting the at least one transparent conductive film 3 with the power supply unit 102 and the switch unit 101, with providing the power supply unit 102 and the switch unit 101 on the pre-processed area (i.e., left side, right side or both) of the snow goggles frame 1.

Still referring to FIGS. 1 through 4, by way of example, the manufacturing method of snow goggles device of the present invention further includes the step S4: manually, semi-automatically or automatically operating the power supply unit 102 to supply an electric current to the transparent conductive film 3 via the switch unit 101 to thereby heat the optical substrate layer 2 for preventing the optical substrate layer (or snow goggles frame 1) turning into a frozen state so as to provide a characteristic of anti-frozen function, anti-frosted function or anti-fog function.

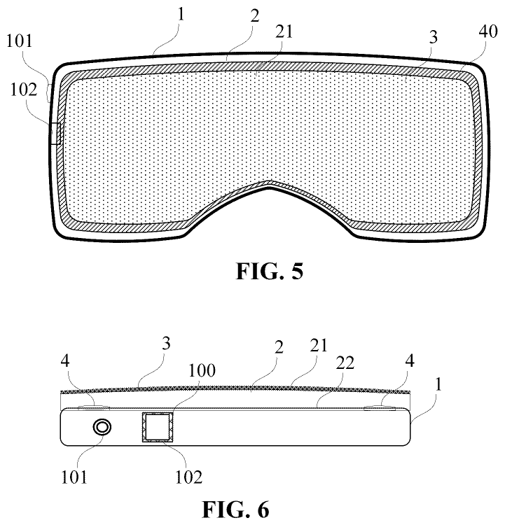

FIG. 5 is a schematic front view of a snow goggles device having a passively defrosting thin film in accordance with a second preferred embodiment of the present invention corresponding to the snow goggles device shown in FIGS. 2 and 3. Turning now to FIG. 5, by way of example, in comparison with the first embodiment, the snow goggles device of the second preferred embodiment of the present invention includes a snow goggles frame 1, an optical substrate layer 2, a transparent conductive film (passively defrosting ultra-thin film) 3, at least one frame-heating unit 40, a switch unit 101 and a power supply unit 102.

Still referring to FIG. 5, by way of example, the frame-heating unit 40 is provided to surround a predetermined periphery of the optical substrate layer 2, with preferably engaging with a predetermined portion between the first surface 21 and the second surface 22, with the frame-heating unit 40 selecting from a frame-heating member, a heating wire member, a transparent heating member (i.e., thin film) or combinations thereof.

FIG. 6 is a schematic side view of a snow goggles device having the passively defrosting thin film in accordance with a fourth preferred embodiment of the present invention. Turning now to FIG. 6, by way of example, in comparison with the second embodiment, the snow goggles device of the fourth preferred embodiment includes a snow goggles frame 1, at least one compartment 100 and at least one lid (not shown) in which to contain the switch unit 101 and the power supply unit 102.

Still referring to FIG. 6, by way of example, in another embodiment, the switch unit 101 and the power supply unit 102 vare coated by a layer of strengthening material in and around the compartment 100 for reinforcing entire structure.

Although the invention has been described in detail with reference to its presently preferred embodiment, it will be understood by one of ordinary skills in the art that various modifications can be made without departing from the spirit and the scope of the invention, as set forth in the appended claims.

What is claimed is:

1. A snow goggles device comprising:
an optical substrate layer provided with a first surface and a second surface;
a snow goggles frame provided with a predetermined area, with providing the optical substrate layer on the predetermined area of the snow goggles frame;
at least one transparent conductive film provided on the first surface, the second surface or therebetween;
at least one power supply unit electrically connecting with the at least one transparent conductive film, with providing the power supply unit on the snow goggles frame; and
a switch unit electrically connecting between the at least one transparent conductive film and the power supply unit, with providing the switch unit on the snow goggles frame;
wherein the power supply unit is operated to supply a current to the at least one transparent conductive film via the switch unit to thereby heat the optical substrate layer for preventing the optical substrate layer turning into a frozen state.

2. The snow goggles device as defined in claim 1, wherein the transparent conductive film further includes at least one auxiliary heater unit which is selected from a frame-heating member, a heating wire member, a transparent heating film member or combinations thereof.

3. The snow goggles device as defined in claim 2, wherein the auxiliary heater unit is integrally formed on a predetermined portion of the snow goggles frame to form a protruded or recessed portion thereon.

4. The snow goggles device as defined in claim 1, wherein the snow goggles frame is further provided with a compartment to receive the at least one power supply unit and the switch unit.

5. The snow goggles device as defined in claim 1, wherein the optical substrate layer is provided with a side peripheral surface between the first surface and the second surface to engage with an auxiliary heater unit.

6. The snow goggles device as defined in claim 1, wherein the transparent conductive film is made of a transparent conductive material or a mixture of transparent conductive material and thermochromic dye material.

7. The snow goggles device as defined in claim 1, wherein the transparent conductive film further includes at least one auxiliary heater unit provided with an illumination member.

8. The snow goggles device as defined in claim 1, wherein the power supply unit electrically connects with at least one illumination member.

9. The snow goggles device as defined in claim 8, wherein the at least one illumination member is selected from a LED element, a solar-cell illumination element, a florescent reflection element or combinations thereof.

10. The snow goggles device as defined in claim 1, wherein the switch unit electrically connects with at least one illumination member.

11. A manufacturing method of a snow goggles device comprising:
providing an optical substrate layer on a snow goggles frame;
providing the optical substrate layer with a first surface and a second surface and providing at least one transparent conductive film on the first surface, the second surface or therebetween;
electrically connecting the at least one transparent conductive film with a power supply unit and a switch unit, with providing the power supply unit and the switch unit on the snow goggles frame; and
operating the power supply unit to supply a current to the at least one transparent conductive film via the switch unit to thereby heat the optical substrate layer for preventing the optical substrate layer turning into a frozen state.

12. The method as defined in claim 11, wherein the transparent conductive film further includes at least one auxiliary heater unit which is selected from a frame-heating member, a heating wire member, a transparent heating film member or combinations thereof.

13. The method as defined in claim 12, wherein the auxiliary heater unit is integrally formed on a predetermined portion of the snow goggles frame to form a protruded or recessed portion thereon.

14. The method as defined in claim 11, wherein the snow goggles frame is further provided with a compartment to receive the at least one power supply unit and the switch unit.

15. The method as defined in claim 11, wherein the optical substrate layer is provided with a side peripheral surface between the first surface and the second surface to engage with an auxiliary heater unit.

16. The method as defined in claim 11, wherein the transparent conductive film is made of a transparent conductive material or a mixture of transparent conductive material and thermochromic dye material.

17. The method as defined in claim 11, wherein the transparent conductive film further includes at least one auxiliary heater unit provided with an illumination member.

18. The method as defined in claim 11, wherein the power supply unit electrically connects with at least one illumination member.

19. The method as defined in claim 18, wherein the at least one illumination member is selected from a LED element, a solar-cell illumination element, a florescent reflection element or combinations thereof.

20. The method as defined in claim 11, wherein the switch unit electrically connects with at least one illumination member.
